(19) United States
(12) Patent Application Publication
(10) Pub. No.: US 2021/0369145 A1
(43) Pub. Date: Dec. 2, 2021

(54) BRACELET AND NECKLACE SAVIOR

(71) Applicants: Pablo Hugo Marcos, Coram, NY (US); America Exolina Marcos, Coram, NY (US)

(72) Inventors: Pablo Hugo Marcos, Coram, NY (US); America Exolina Marcos, Coram, NY (US)

(21) Appl. No.: 16/885,181

(22) Filed: May 27, 2020

(51) Int. Cl. A61B 5/11; A41D 31/04; A44C 5/00; A44C 15/00

(52) U.S. Cl. CPC A61B 5/1127; A61B 5/1121; A41D 31/04; A61B 2562/16; A44C 15/005; A61B 2562/0257; A44C 5/0007

(57) ABSTRACT

An article of manufacture for providing a wearable face and hand proximity detector is disclosed. The article includes a bracelet to be worn on a user's wrist and a necklace to be worn around the user's neck. One of these devices includes a signal transmitter that may be received by a receiver element in the other device. The received signal being detected provides an indication that the user's hand is nearing the user's face. When the signal is detected, an alarm is generated. The alarm may be an auditory signal or a physical vibration that may be felt by the user.

### TECHNICAL FIELD

This application relates in general to an article of manufacture for providing a personal protection device, and more specifically, to an article of manufacture for providing a wearable face and hand proximity detector.

### BACKGROUND

In these terrible days of turmoil, grief and uncertainty due to the covid-19 virus, our society needs something to help change certain physical habits. Something as simple as touching our faces with our hands can be critical. Individuals typically do not recognize how often they allow their hands to touch parts of their faces. This behavior is an instinctive or subconscious action. The repeated touching of one's face may lead to the spread of viruses, bacteria, and similar pathogens. This behavior may be eliminated if a person is repeatedly warned each time his/her hand approaches his/her face. Any device that may warn individuals needs to be unobtrusive to permit it to be discretely worn while training to eliminate the behavior.

Therefore, a need exists for a small electronic device to act as a wearable face and hand proximity detector. The detector may be embedded into items easily worn by users who wish to train themselves not to engage in this behavior.

### SUMMARY

In accordance with the present invention, the above and other problems are solved by providing an article of manufacture for a wearable face and hand proximity detector according to the principles and example embodiments disclosed herein.

In one embodiment, the present invention is an article of manufacture for providing a wearable face and hand proximity detector. The detection device is a demi parure that includes a bracelet to be worn on a user's wrist and a necklace to be worn around the user's neck. One of these jewelry items includes a signal transmitter that may be received by a receiver element in the other item. The signal provides an indication that the user's hand is nearing the user's face. An alarm is generated when the signal is detected . The alarm may be an auditory signal or a physical vibration that may be felt by the user.

The foregoing has outlined rather broadly the features and technical advantages of the present invention in order that the detailed description of the invention that follows may be better understood. Additional features and advantages of the invention will be described hereinafter that form the subject of the claims of the invention.

It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features that are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only, and is not intended as a definition of the limits of the present invention.

### BRIEF DESCRIPTION OF THE DRAWINGS

Referring now to the drawings in which like reference numbers represent corresponding parts throughout:

FIG. 1 illustrates an example embodiment for a user wearing a Bracelet and Necklace Savior according to the present invention.

FIG. 2 illustrates an example embodiment of jewelry items that provide a wearable face and hand proximity detector according to the present invention.

FIG. 3 illustrates an example embodiment of electronics within the jewelry items that provide a wearable face and hand proximity detector according to the present invention.

### DETAILED DESCRIPTION

This application relates in general an article of manufacture for providing a personal protection device, and more specifically, to an article of manufacture for providing a wearable face and hand proximity detector according to the present invention.

Various embodiments of the present invention will be described in detail with reference to the drawings, wherein like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

In describing embodiments of the present invention, the following terminology will be used. The singular forms “a,” “an,” and “the” include plural referents unless the context clearly dictates otherwise. Thus, for example, reference to “a needle” includes reference to one or more of such needles and “etching” includes one or more of such steps. As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. As used herein, the singular forms “a,” “an,” and “the” are intended to include the plural forms as well, unless the context clearly indicates otherwise.

It further will be understood that the terms “comprises,” “comprising,” “includes,” and “including” specify the presence of stated features, steps or components, but do not preclude the presence or addition of one or more other features, steps or components. It also should be noted that in some alternative implementations, the functions and acts noted may occur out of the order noted in the figures. For example, two figures shown in succession may in fact be executed substantially concurrently or may sometimes be executed in the reverse order, depending upon the functionality and acts involved.

As used herein, the term “about” means that dimensions, sizes, formulations, parameters, shapes, and other quantities and characteristics are not and need not be exact, but may be approximated and/or larger or smaller, as desired, reflecting tolerances, conversion factors, rounding off, measurement error and the like, and other factors known to those of skill. Further, unless otherwise stated, the term “about” shall expressly include “exactly,” consistent with the discussion above regarding ranges and numerical data.

The terms “customer,” and “user” refer to an entity, e.g. a human, using the Bracelet and Necklace Savior associated with the invention. The term user herein refers to one or more users.

The term “invention” or “present invention” refers to the invention being applied for via the patent application with the title “Bracelet and Necklace Savior.” Invention may be used interchangeably with detector.

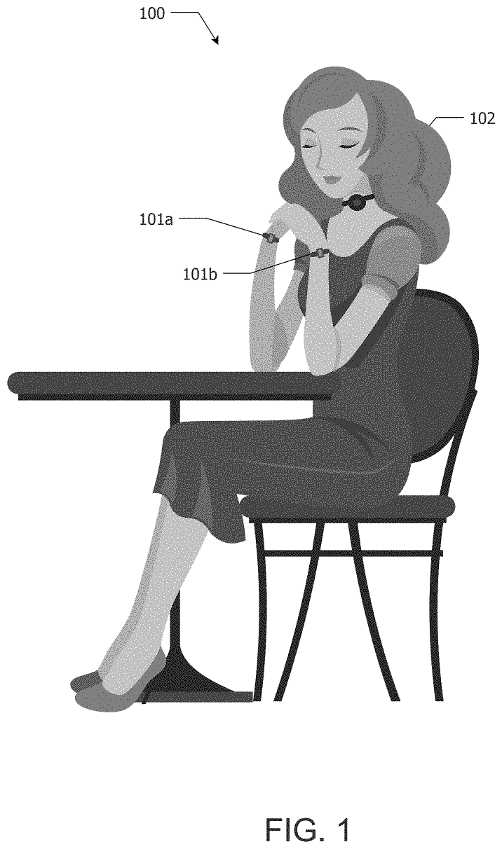

In general, the present disclosure to an article of manufacture for providing a personal protection device. To better understand the present invention, FIG. 1 illustrates an example embodiment for a user wearing a Bracelet and Necklace Savior set according to the present invention. A user 100 is shown engaging in everyday activities. Her hands are routinely moving about her in front of her face and body. The user 100 is shown wearing a bracelet 101 and a necklace 102 as ordinary articles of jewelry.

The necklace 102 and a bracelets 101a-b on either hand may be configured to provide an alarm, such as to vibrate or to emit a sound, when the device detects that the user's hand approaches her face. Battery-powered electronics within both the necklace 102 and the bracelet 101 provide a combination of electro-magnetic sensors in each device.

The necklace 102 and a bracelets 101a-b may be offered in an array of materials. The jewelry items may be offered at different levels material and design, Entry level units primarily will be made of plastic and fabric. Both the necklace and the bracelet may be adjustable. The set may be comprised of a variety of colors and materials. Major jewelry manufacturers and accessories companies may manufacture an assortment of designs and finishes to make the devices attractive to wear.

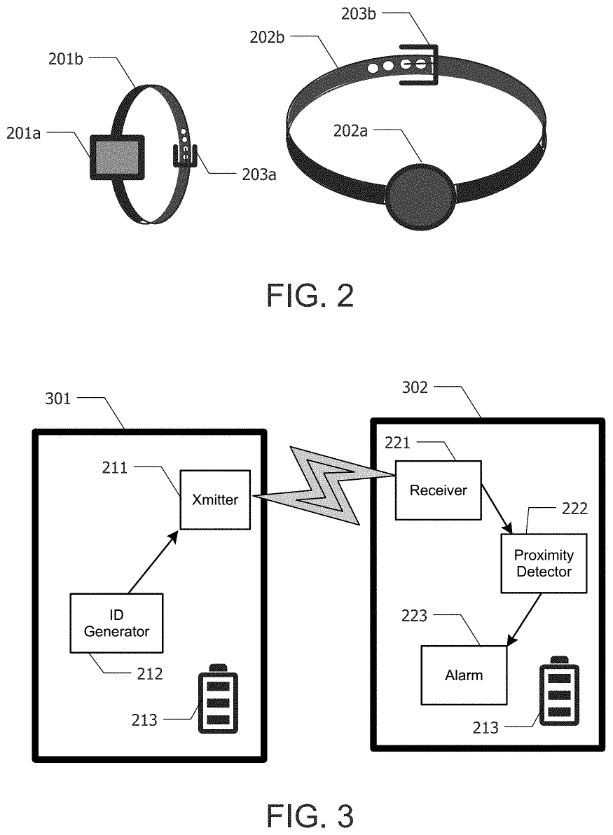

FIG. 2 illustrates an example embodiment of a jewelry item that provides a wearable face and hand proximity detector according to the present invention. The necklace 102 and one bracelet 101a are shown having adjustable bands 203a-b and electronic enclosures 204a-b. The user wears each item 101-102 in the typical manner with the adjustable bands 203a-b arranged to hold the devices 101-102 on the user's body. The electronic enclosures 204a-b also may be adorned with items to improve their appearance.

The adjustable bands 203a-b are shown to utilize a buckle to hold the bands at a desired length. Other types of mechanisms, such as Velcro™, clasps, clips, and the like may also be used to secure the straps. Additionally, the necklace 102 may be worn on a chain or styled as a lanyard to place its electronic enclosure high enough to be near the user's face.

FIG. 3 illustrates an example embodiment of electronics in jewelry items that provide a wearable face and hand proximity detector according to the present invention. The electronics within the bracelets 101a-b and necklace 102 consist of a transmitter module 301 and a receiver module 302. In a preferred embodiment, a transmitter module 301 is placed into one of the two jewelry items 101-102 and the receiver module 302 is placed into the other item. In other embodiments, both a transmitter module 301 and a receiver module 302 may be placed in all of the jewelry items 101-102.

The transmitter module 301 consists of a transmitter 311, an ID generator 312, and a battery 213 that is used to power all of the electronics within the jewelry 101-102. The transmitter module 301 sends an electro-magnetic signal to be detected and used by the receiver module 302. The transmitter 311 may send its signal in a radio frequency (RF) and/or via infrared radiation (IR) . The transmitter module 301 uses the ID generator 312 to generate a unique ID to be encoded and transmitted by the transmitter 311 so that the receiver module may distinguish between multiple devices. The transmitter module 301 and the receiver module 302 may use a matching process to identify the unique ID to be sent by its corresponding device.

The receiver module 302 consists of a receiver 321, a proximity detector 322, and an alarm 323. The receiver module 302 also includes its own battery 213 to power all of the electronics in the receiver module 302. The receiver 322 receives a transmitted signal from the transmitter 311 and sends the received signal to the proximity detector 322 for processing. If the transmitted signal includes a unique ID, the proximity detector 322 may decode the transmitted digital bit stream into a received ID code. When the received ID code matches the unique ID sent by the transmitter 311, the proximity detector 322 determines that the received signal is from its mated device. The proximity detector 322 generates a signal that is sent to the alarm 323 when the proximity detector 322 determines that the transmitter module 301 and the receiver module 302 are too close together. In one embodiment, the proximity detector 322 generates the alarm signal when the received signal has a signal amplitude above a predetermined level.

The alarm 323 receives the signal from the proximity detector 322 and generates an auditory and/or a vibrating signal to the user. The alarm 323 may generate either type of alarm to the user or may generate both signals depending upon the embodiment used. The user will hear and feel the alarm to indicate that his/her hand is nearing his/her face. Repeated alarms from the devices will train the user to realize how often his/her hand(s) near his/her face and to act to stop that behavior before any pathogens can be passed from the hand to the nose or mouth.

While the embodiments herein present two bracelets and a necklace, other embodiments are also possible. For example, a configuration with one bracelet and one necklace is possible. In another embodiment, the system may use two bracelets worn on the wrists and two corresponding pairs of devices are located within earrings if the electronics and battery may be made to fit into an acceptably sized earring. Other embodiments using head bands, caps , hats, may also be used. These variations may be specifically relevant when the devices are worn by children rather than an adult, or is worn by a boy instead of a girl. The scope of the present invention should be limited only by the language in the claims attached herein.

Even though particular combinations of features are recited in the present application, these combinations are not intended to limit the disclosure of the invention. In fact, many of these features may be combined in ways not specifically recited in this application. In other words, any of the features mentioned in this application may be included to this new invention in any combination or combinations to allow the functionality required for the desired operations.

No element, act, or instruction used in the present application should be construed as critical or essential to the invention unless explicitly described as such. Further, the phrase “based on” is intended to mean “based, at least in part, on” unless explicitly stated otherwise. Any singular term used in this present patent application is applicable to its plural form even if the singular form of any term is used.

What is claimed:

1. An article of manufacture for providing a wearable face and hand proximity detector, the article comprises:
one or more transmitter devices located in a first wearable item, the one or more transmitter devices comprise:
a signal transmitter for emitting a known location detection signal; and
a battery; and
one or more receiver devices located in a second wearable item, the one or more receivers comprise:
a receiver for receiving the known location detection signal;
a proximity detector for detecting when the one or more transmitter devices are near the one or more receiver device;
an alarm for generating an alarm indication that the proximity detector has detected the one or more transmitter devices are near the one or more receiver device; and
a battery.

2. The article of manufacture according to claim 1, wherein the first wearable item is a necklace worn just below a face; and
the second wearable item is a bracelet worn on a wrist.

3. The article of manufacture according to claim 2, wherein the one or more receiver devices corresponds to two bracelets worn on each of a user's wrists.

4. The article of manufacture according to claim 1, wherein the second wearable item is a necklace worn just below a face; and
the first wearable item is a bracelet worn on a wrist.

5. The article of manufacture according to claim 4, wherein the one or more transmitter devices corresponds to two bracelets worn on each of a user's wrists.

6. The article of manufacture according to claim 1, wherein the transmitter emits an RF signal.

7. The article of manufacture according to claim , wherein an RF signal corresponds to a Bluetooth signal.

8. The article of manufacture according to claim 1, wherein the transmitter emits an IF signal.

9. The article of manufacture according to claim 1, wherein the one or more transmitter devices further comprise an ID generator for uniquely identifying the first wearable item location, the ID generator creates a unique ID for transmission within the known location detection signal.

10. The article of manufacture according to claim 9, wherein. the proximity detector uses the unique ID to determine whether the alarm generates the alarm indication.

11. The article of manufacture according to claim 1, wherein the alarm indication is a physical vibration.

12. The article of manufacture according to claim 1, wherein the alarm indication is an audible sound.
